(19) United States
(12) Patent Application Publication
(10) Pub. No.: US 2023/0236196 A1
(43) Pub. Date: Jul. 27, 2023

(54) SARS-COV-2 INFECTION RISK ASSESSMENT METHOD

(71) Applicants: VIROGATES A/S, Birkerod (DK); COPENHAGEN UNIVERSITY HOSPITAL AMAGER AND HVIDOVRE, Hvidovre (DK)

(72) Inventors: Jesper Eugen-Olsen, Birkerod (DK); Ove Andersen, Hvidovre (DK)

(21) Appl. No.: 17/916,863

(22) PCT Filed: Apr. 7, 2021
(86) PCT No.: PCT/EP2021/059107

§ 371 Date: Oct. 4, 2022

(30) Foreign Application Priority Data
Apr. 8, 2020 (GB) 2005232.0
Apr. 23, 2020 (GB) 2005977.0

(51) Int. Cl. G01N 33/68

(52) U.S. Cl. CPC G01N 33/6827; G01N 33/6893; G01N 2333/9723; G01N 2800/56

(57) ABSTRACT

Increased levels of soluble urokinase-type plasminogen activator receptor (suPAR), particularly a plasma level of over 4.75 ng/ml or 6 ng/nl, have been found to be a predictor of whether a subject with COVID-19 symptoms and/or SARS-CoV-2 infection will require oxygen supplementation.

### BACKGROUND AND FIELD OF THE INVENTION

The invention concerns the examination of subjects with symptoms of COVID-19.

The COVID-19 pandemic caused by the corona virus SARS-CoV-2 puts tremendous pressure on hospital resources and capacity. Risk triaging is important in order to quickly discharge those patients who will not develop severe disease to own home isolation and admit those who will develop more severe disease to the medical ward, or to the Intensive Care Unit (ICU).

In Denmark, the rate of infection is rapidly increasing but at time of submission of this patent, the hospital resources and capacity were able to accommodate those with signs of severe infection. However, those with milder symptoms that are sent home may also progress to more severe disease and need of respiratory assistance and, in worst case, die at home. In addition, in other countries such as Italy, hospital resources are overstretched and it would be very useful to identify, with improved accuracy, those who are most at need of admittance, particularly to the ICU.

Therefore, a biomarker that can predict the disease progression is useful in discharging patients, also those with moderate disease if risk of further progression is low and they can be safely released for home quarantine.

Some risk markers for development of serious illness and death caused by SARS-CoV-2 are already known, for example age and sex. The risk of complications and death rises with age, particularly in those over 65, and is greater in men than women. Certain underlying health conditions are also known to increase risk, particularly cancer, severe obesity, immunosuppression (for example arising from anti-cancer chemotherapy or a low CD4 cell count), diabetes, hypertension, heart conditions, lung conditions (such as asthma), liver disease, kidney disease, and possibly certain neurological conditions such as motor neurone disease. An AI-based analysis has also suggested that having a combination of increased levels of the liver enzyme alanine aminotransferase (ALT), reported myalgia (deep muscle aches), and increased haemoglobin levels is a risk factor.

It has now been found that an increased level of a protein termed suPAR is a risk factor for the severity of the disease and death. The protein suPAR (NCBI Accession no. AAK31795 and isoforms of the receptor, NP_002650, 003405, NP_002650, NP_OO1005376) is the soluble portion of urokinase-type Plasminogen Activator Receptor (uPAR), which is released by cleavage of the GPI anchor of membrane-bound uPAR. suPAR is a family of glycosylated proteins consisting of full length suPAR (277 amino acids (1-277)) and suPAR fragments D1 (1-83), and D2D3 (84-277) generated by urokinase cleavage or human airway trypsin-like protease, D1 (1-87) and D2D3 (88-277) generated by MMP cleavage, D1 (1-89) and D2D3 (90-277) also generated by urokinase cleavage or human airway trypsin-like protease, D1 (1-91) and D2D3 (92-277) generated by cleavage by plasmin. Continuous and discontinuous epitopes present in the protein suPAR and its cleavage products may be used to monitor their presence and abundance in a biological fluid by immunodetection with mono- or polyclonal antibodies. Antibodies directed to accessible epitopes common to suPAR and its cleavage products (e.g. D2D3) can be used to detect both suPAR and its cleavage products in a biological fluid. Since there is a one-to-one relationship between suPAR and its cleavage products, an antibody that is directed to an epitope that is common to both full length suPAR and, say, the D2D3 cleavage product will at the same time directly and indirectly measure the suPAR level. That is to say, a value of, say, 3 ng/ml as measured in the assay is regarded as indicating a suPAR level of 3 ng/ml, even though some of the protein that was detected may have been the D2D3 cleavage product. In the context of the assay, therefore, “suPAR” refers to full length suPAR and its cleavage product D2D3. The term D2D3 is used to denote any suPAR-derived fragment corresponding to the 84-277 region of suPAR and having an N-terminus lying in the 84-92 amino acid region of suPAR and a C-terminus corresponding to the C-terminus of suPAR (amino acid 277), for example 84-277, 88-277, 90-277 and 92-277.

WO 2008/077958 (Hvidovre Hospital) discloses the use of suPAR as a biomarker for low-grade inflammation (LGI), diseases associated with LGI, and metabolic syndrome. It also discloses the measurement of suPAR levels in apparently healthy subjects as a means of assessing the risk of developing a disease (such as cardiovascular disease) and the overall risk of mortality within ten years, principally so that lifestyle changes can be made in order to reduce those risks. Determining the risk of developing a disease (as opposed to having the disease) and the risk of mortality within ten years in an apparently healthy subject is not relevant to the sort of assessments that are needed in an ED.

suPAR has also previously been shown to be a biomarker of readmission to hospital and of mortality in acute medical patients (WO 2019/162334). However, suPAR has never been investigated in patients with symptoms of, or verified, COVID-19, nor has it been investigated with the endpoint of need of respiratory assistance (e.g. non-invasive ventilation (NIV) or continuous positive airway pressure (CPAP) or respirator).

### SUMMARY OF THE INVENTION

The present invention aims to provide a novel means by which medical personnel can (optionally in conjunction with other clinical observations and medical history etc) assess the state of a subject and, in particular, the subject's risk of needing non-invasive ventilation (NIV) or continuous positive airway pressure (CPAP) or a respirator. This enables more accurate assessments to be made concerning whether a subject should be admitted to hospital or discharged.

In particular, the examination concerns the measuring of a protein termed (suPAR) in a body fluid, particularly blood samples, with the aim of determining whether the patient needs oxygen supplementation or not.

The subject is considered to have a fever if their temperature is over 37° C. (for example assessed by an oral, rectal or armpit thermometer or a non-contact thermometer, for example aimed at the forehead or interior of the ear) or if the subject's forehead or back feel hot.

A new, continuous, cough is one that involves coughing for more than an hour, or three or more coughing episodes in 24 hours.

We have found that

1. Patients presenting with symptoms of COVID-19 patients with elevated suPAR (above 4.75 ng/ml and particularly above 6 ng/ml) have significantly increased risk of requiring respiratory assistance in near future (within 14 days) compared with COVID-19 patients with suPAR below 4.75 ng/ml or 6 ng/ml, respectively.

2. In patients that are in need of continuous positive airway pressure (patients receiving continuous positive airway pressure, CPAP) or patients receiving invasive ventilation in a respirator (invasive ventilation is positive pressure ventilation applied via an endotracheal or tracheotomy tube), the suPAR kinetics can be used to determine whether the patients will survive or not.

Thus, the invention also provides a method of determining the likelihood of death within a period of time of a subject who has COVID-19 symptoms and/or SARS-CoV-2 infection and is being treated with, or is being considered for treatment with, assisted respiration, the method comprising determining whether the subject has a suPAR blood level of over 4.75 ng/ml, particularly over 6 ng/ml. In hospitals in which there are insufficient resources, this may enable medical staff to determine who will be most likely to benefit from the assisted ventilation.

The value of 6 ng/ml is expressed to one significant figure and thus may include values from 5.5 ng/ml. Alternatively, “6 ng/ml” means 6.0 ng/ml.

The period of time may be 1, 2, 3, 4, 5, 6, 7, 8, 9, 10, 11, 12, 13 or 14 days.

The assisted ventilation may comprise non-invasive ventilation (NIV), continuous positive airway pressure (CPAP), or invasive mechanical ventilation.

If the subject's suPAR level is over 20 ng/ml, then urgent assisted ventilation is needed, i.e. within the next 30 minutes, 1 hour, 2 hours, 3 hours or 4 hours.

Non-invasive ventilation can, for example be continuous positive airway pressure (CPAP), which is a type of positive airway pressure, where the air flow is introduced into the airways to maintain a continuous pressure to constantly stent the airways open, in people who are breathing spontaneously. Positive end-expiratory pressure (PEEP) is the pressure in the alveoli above atmospheric pressure at the end of expiration. CPAP is a way of delivering PEEP but also maintains the set pressure throughout the respiratory cycle, during both inspiration and expiration. It is measured in centimeters of water pressure (cm H₂O). Non-invasive ventilation can alternatively be bilevel positive airway pressure (BiPAP) where the pressure delivered differs based on whether the patient is inhaling or exhaling. These pressures are known as inspiratory positive airway pressure (IPAP) and expiratory positive airway pressure (EPAP). In CPAP no additional pressure above the set level is provided, and patients are required to initiate all of their breaths.

Invasive mechanical ventilation can become a lifesaving intervention for patients with respiratory and breathing difficulties. The term “invasive” is used if it involves any instrument penetrating via the mouth (such as an endotracheal tube), nose, or the skin (such as a tracheostomy tube through a stoma, a surgically-created hole in the windpipe) to serve as an artificial airway.

### EXAMPLES

### Example 1—Determination of Fever

Fever may be determined as a temperature of over 37° C., as assessed by an oral, rectal or armpit thermometer, or a non-contact forehead thermometer, or if the subject's forehead or back feel hot.

### Example 2 Determination of Cough

A new continuous cough is defined as a cough that persists for more than hour or that has more than three coughing episodes in 24 hours.

### Example 3—Diagnosis of SARS-CoV-2 Infection

A diagnosis of a SARS-CoV-2 infection can be achieved by means of any test, for example those in the following table (derived from the US FDA, 8 Apr. 2020):

TABLE 1

| Manufacturer | Assay |
|---|---|
| InBios International, Inc | Smart Detect SARS-CoV-2 rRT-PCR Kit |
| Gnomegen LLC | Gnomegen COVID-19 RT-Digital PCR |
| | Detection Kit |
| Co-Diagnostics, Inc. | Logix Smart Coronavirus Disease 2019 |
| | (COVID-19) Kit |
| ScienCell Research | ScienCell SARS-CoV-2 Coronavirus Real- |
| Laboratories | time RT-PCR (RT-qPCR) Detection Kit |
| Luminex Corporation | ARIES SARS-CoV-2 Assay |
| Becton, Dickinson & | BioGX SARS-CoV-2 Reagents for BD MAX |
| Company (BD) | System |
| Ipsum Diagnostics, LLC | COV-19 IDx assay |
| Cellex Inc. | qSARS-CoV-2 IgG/IgM Rapid Test |
| QIAGEN GmbH | QIAstat-Dx Respiratory SARS-CoV-2 Panel |
| NeuMoDx Molecular, Inc. | NeuMoDx SARS-CoV-2 Assay |
| Luminex Molecular | NxTAG CoV Extended Panel Assay |
| Diagnostics, Inc. | |
| Abbott Diagnostics | ID NOW COVID-19 |
| Scarborough, Inc. | |
| BGI Genomics Co. Ltd | Real-Time Fluorescent RT-PCR Kit for |
| | Detecting SARS-2019-nCoV |
| Avellino Lab USA, Inc. | AvellinoCoV2 test |
| PerkinElmer, Inc. | PerkinElmer New Coronavirus Nucleic Acid |
| | Detection Kit |
| Mesa Biotech Inc. | Accula SARS-Cov-2 Test |
| BioFire Defense, LLC | BioFire COVID-19 Test |
| Cepheid | Xpert Xpress SARS-CoV-2 test |
| Primerdesign Ltd. | Primerdesign Ltd COVID-19 genesig Real- |
| | Time PCR assay |
| GenMark Diagnostics, Inc. | ePlex SARS-CoV-2 Test |
| DiaSorin Molecular LLC | Simplexa COVID-19 Direct assay |
| Abbott Molecular | Abbott RealTime SARS-CoV-2 assay |
| Quest Diagnostics | Quest SARS-CoV-2 rRT-PCR |
| Infectious Disease, Inc. | |
| Quidel Corporation | Lyra SARS-CoV-2 Assay |
| Laboratory Corporation of | COVID-19 RT-PCRTest |
| America (LabCorp) | |
| Hologic, Inc. | Panther Fusion SARS-CoV-2 |
| Thermo Fisher Scientific, | TaqPath COVID-19 Combo Kit |
| Inc. | |
| Roche Molecular Systems, | cobas SARS-CoV-2 |
| Inc. (RMS) | |
| Wadsworth Center, New | New York SARS-CoV-2 Real-time Reverse |
| York State Department of | Transcriptase (RT)-PCR Diagnostic Panel |
| Public Health's (CDC) | |
| Centers for Disease Control | CDC 2019-nCoV Real-Time RT-PCR |
| and Prevention's (CDC) | Diagnostic Panel (CDC) |

### Example 4—Measurement of suPAR Level

suPAR levels may be measured in body fluids by the methods taught in WO 2008/077958, which is incorporated herein for that purpose.

More specifically, suPAR levels may be determined by ELISA assay as follows: Nunc

Maxisorp ELISA-plates (Nunc, Roskilde, Denmark) are coated overnight at 4° C. with a monoclonal rat anti-suPAR antibody (VG-1, ViroGates A/S, Copenhagen, Denmark, 3 μg/ml, 100 μI/well). Plates are blocked with PBS buffer+1% BSA and 0.1% Tween 20, 1 hour at room temperature, and washed 3 times with PBS buffer containing 0.1% Tween 20. 85 μI dilution buffer (100 mm phosphate, 97.5 mm NaCl, 10 g L⁻¹ bovine serum albumin (BSA, Fraction V, Roche Diagnostics GmbH Penzberg, Germany), 50 U mL⁻¹ heparin sodium salt (Sigma Chemical Co., St. Louis, Mo.), 0.1% (v/v) Tween 20, pH 7.4) containing 1.5 μg/ml HRP labeled mouse anti-suPAR antibody (VG-2-HRP, ViroGates) and 15 μI plasma (or serum or urine) sample is added in duplicates to the ELISA plate. After 1 hour of incubation at 37° C., plates are washed 10 times with PBS buffer+0.1% Tween 20 and 100 μI/well HRP substrate added (Substrate Reagent Pack, R&D Systems Minneapolis, Minn.). The colour reaction is stopped after 30 min using 50 μI per well 1M H₂SO₄ and measured at 450 nm.

Furthermore, suPAR can be measured in bodily fluids using commercially available CE/IVD approved assays such as the suPARnostic® product line according to the manufacturer's instructions. In the TRIAGE III trials, suPAR was quantified using the suPARnostic Quick Triage lateral flow assay.

The suPAR level may, for example, be assayed using the suPARnostic® Autoflex ELISA test sold by ViroGates A/S, Banevnget 13, DK-3460 Birkerød, Denmark. Alternatively, suPAR levels can be measured by proteomic approaches such as western blot, Luminex, MALDI-TOF, HPLC or Genspeed device and automated immune analyzer platforms such as Bayer Centaur, Abbott Architect, Abbott AxSym, Roche COBAS and the Axis Shield Afinion or using turbidimetric assays such as suPARnostic® Turbilatex on Roche, Cobas c111, Cobas c501/2+c701/2, or Siemens ADVIA XPT or Centaur or Abbott Architect.

Monoclonal antibodies to the said receptor or receptor peptides used in the method of the present invention may be prepared using any technique which provides for the production of antibody molecules by continuous cell lines in culture. These include, but are not limited to, the hybridoma technique, the human B-cell hybridoma technique, and the EBV-hybridoma technique. See, e.g., Kohler, et al, 1975, Nature 256: 495-497; Kozbor et al, 1985, J. Immunol. Methods 81: 31-42; Cote et al, 1983, Proc. Natl. Acad. Sci. USA 80: 2026-2030; Cole et al, 1984, Mol. Cell Biol. 62: 109-120. Specifically, the method comprises the following steps: (a) immunizing an animal with an immunogenic receptor peptide; (b) isolating antibody producing cells from the animal; (c) fusing the antibody producing cells with immortalized cells in culture to form monoclonal antibody-producing hybridoma cells; (d) culturing the hybridoma cells; and (e) isolating from the culture monoclonal antibodies which bind to said polypeptide.

The suPAR level in blood may be measured directly in a blood sample or in serum, plasma or urine. Anticoagulant plasma is preferred e.g. EDTA or Citrate plasma. A plasma level over 4.75 ng/ml (especially over 6 ng/ml) is considered to indicate that the subject will require, or is likely to require, oxygen supplementation, in particular with invasive ventilation.

Where the biological sample is urine, the measurements may be based on the urine suPAR/creatinine value from a subject, since this value is known to be highly correlated to the concentration of suPAR in a plasma sample derived from the same subject.

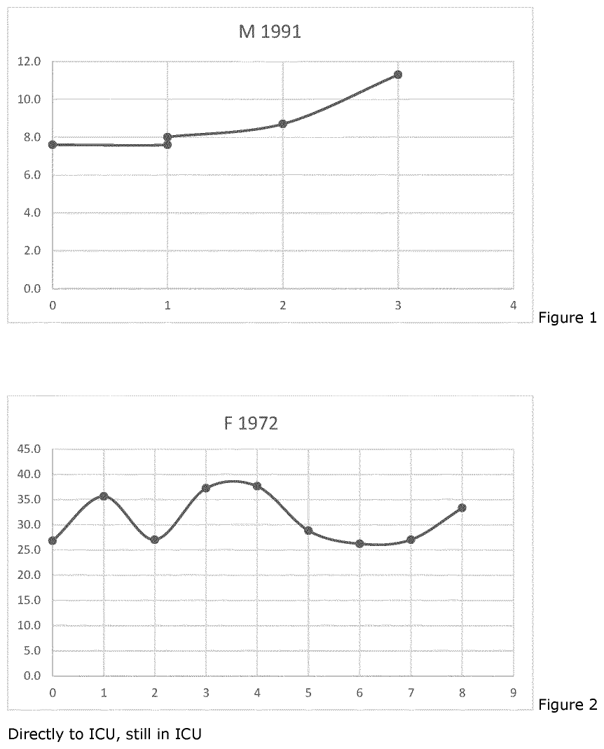

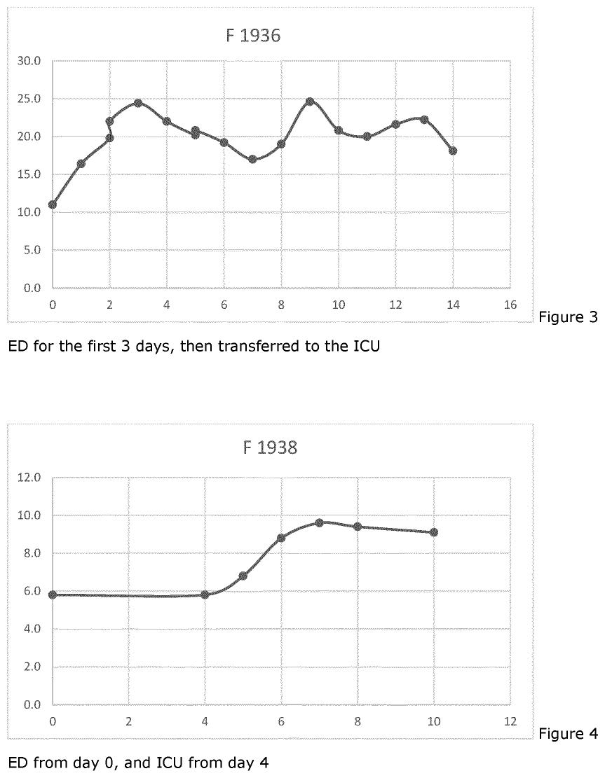

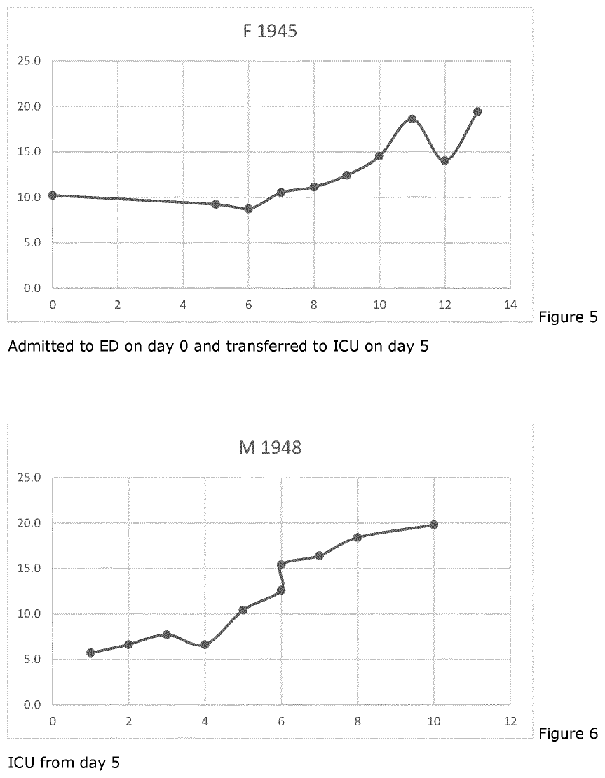

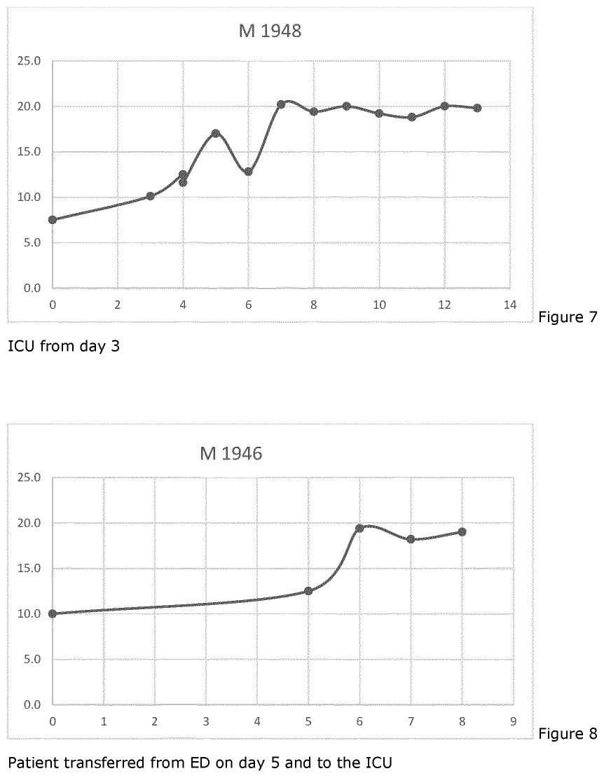

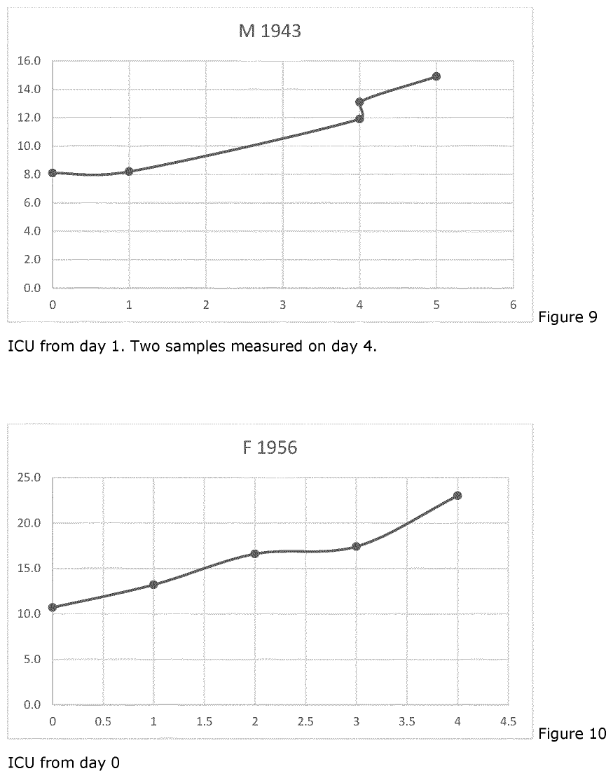

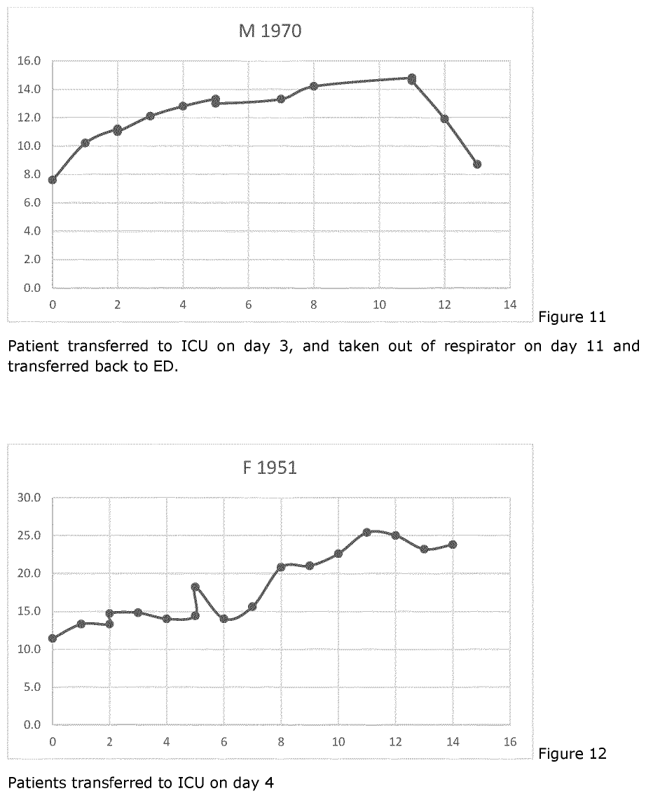

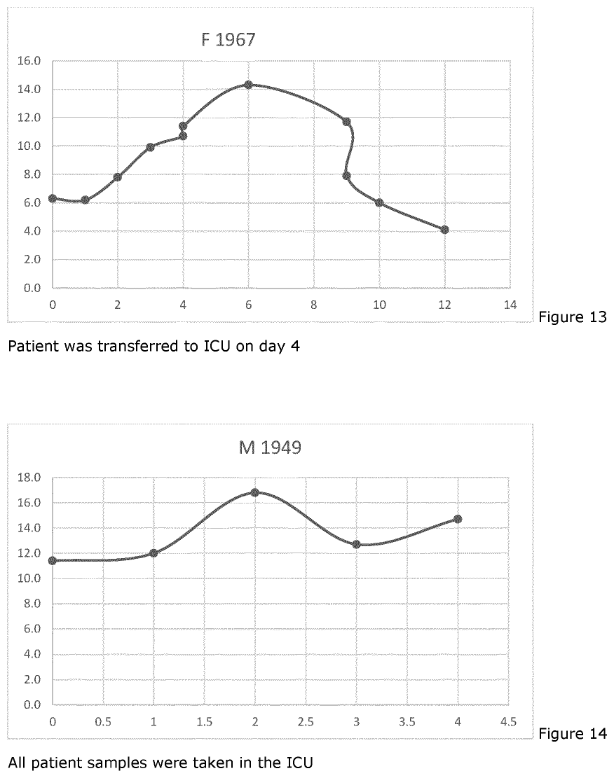

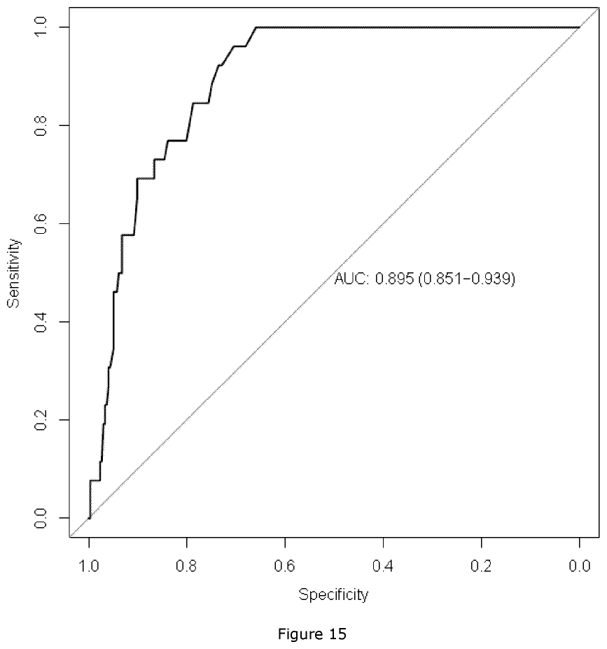

Thus, urine samples may also be employed for the measurement of suPAR, where the measured level in urine is normalized for protein content (e.g. using creatinine). These normalized values may be employed as a marker for the purposes of the present invention. See Example 2 and FIG. 1 of WO 2019/162334.

### Example 5—Clinical Results

### Methods:

The measurement of suPAR in patients was approved by the Danish Health and Medicines Authority (ref. 3-3013-1061/2) and the Danish Data Protection Agency (ref. HVH-2014-018, 02767).

The prospective study was conducted at Copenhagen University Hospital Hvidovre in Denmark. Patients with suspected COVID-19 were included.

C-reactive protein (CRP) was measured using a COBAS 6000 analyzer (Roche Diagnostics, Mannheim, Germany.

suPAR measurements: Blood (EDTA, 4 ml) was drawn on arrival of suspected COVID patients, centrifuged for 2 minutes and suPAR was measured in plasma using a Point-of care test (suPARnostic Quick Triage, ViroGates, Birkerød, Denmark). The test provides a result in 20 minutes, and suPAR was measured real time 24/7. The first patient was included on Mar. 19, 2020 and the last 3^{rd} of April.

442 patients presenting with COVID-19 were included in the study. Of these 179 were male, 250 were female and 3 lacked information on sex.

suPAR was measured at the first presentation in the acute medical department, and patients were followed for up to 18 days. During follow-up, 14 of the patients were admitted to the ICU for either non-invasive ventilation (NIV), e.g. CPAP, or respirator care. The mean suPAR level was significantly higher in patients that ended up in the ICU compared to those that did not (mean 8.7 ng/ml versus 4.6 ng/ml, respectively, p<0.001). This difference was also reflected in the difference in median suPAR (7.85 ng/ml, versus 4.0 ng/ml respectively, p<0.001).

Monitoring of patients in the ICU: Patients in the ICU were measured daily for their suPAR levels, from the period of 19th of March to 3^{rd} of April.

The Figures are suPAR concentrations in individual patients.

In the Figures, the Y-axis shows the suPAR concentration in ng/ml. Note the different axis values.

The X-axis shows the days from first measurements and the number refers to days after the measurement. In a few cases, suPAR was measured twice a day (morning and evening) and thus two data measurements are shown for the same day. Sex and year of birth are given for each patient, for example “M1991” denotes a male born in 1991. ED refers to the COVID Emergency Department. ICU refers to the Intensive Care Unit.

The following list of comorbidities at baseline were noted: None (71, 17,3%), COPD (75, 18,2%), Asthma (64, 15,6%), Diabetes—type 1 (5, 1,2%), Diabetes-type 2 (73, 17,8%), Hypertension (163, 39,7%), Heart failure (52, 12,7%), Diagnosed coronary disease (36, 8,8%), Cancer—active (28, 6,8%), Cancer—non-active (32, 7,8%), Chronic renal failure (21, 5,1%), Chronic liver disease (5, 1,2%), Other lung disease (21, 5,1%), Other heart disease (60, 14,6%), Other chronic infectious disease (5, 1,2%), Other inflammatory disease (17, 4,1%), Alcohol abuse (19, 4,6%). The median number of co-morbidities are shown in Table 2 below.

TABLE 2

| Total | | | | | | | | Percentile | | | | | | |
|---|---|---|---|---|---|---|---|---|---|---|---|---|---|---|
| Count | | | | | | | | | | | 0.50 | | | |
| (N) | Missing | Unique | Min | Max | Mean | StDev | Sum | 0.05 | 0.10 | 0.25 | Median | 0.75 | 0.90 | 0.95 |
| 432 | 0 (0.0%) | 8 | 0.00 | 8.00 | 1.94 | 1.55 | 840.00 | 0.00 | 0.00 | 1.00 | 2.00 | 3.00 | 4.00 | 5.00 |

Information on smoking was available in 405 patients and the distribution was as follows: Active smoker (86, 21,2%), Ex-smoker (150, 37,0%), Never smoked (169, 41,7%)

About travel within the last 14 days, 404 were interviewed and 4,2% reported yes, and 95,8% had not travelled. 10,4% had known contact with another COVID-19 patient, the remaining 362 did not know how they were infected.

At patient presentation, the following symptoms were reported, Sore throat (85, 19,9%), Cough—productive (102, 23,8%), Cough—non-productive (174, 40,7%), Body pain (117, 27,3%), Tired (67, 15,7%), Headache (56, 13,1%), Dizziness (36, 8,4%), Nausea/vomit (53, 12,4%), Fever (217, 50,7%), Abdominal pain (18, 4,2%), Obstipation (0, 0,0%), Diarrhoea (40, 9,3%), Dysuria (6, 1,4%), Dyspnoea (266, 62,1%), Chest pain (55, 12,9%), Arthralgia (8, 1,9%), Cramp (0, 0,0%), Chills (21, 4,9%), Hemoptysis (2, 0,5%) and Other (26, 6,1%). Duration of symptoms: 0-1 days (79, 19,5%), 2-3 days (86, 21,2%), 4-5 days (51, 12,6%), 6-7 days (56, 13,8%), 8-10 days (32, 7,9%), 11-13 days (16, 4,0%), 14-15 days (39, 9,6%), 15+ days (46, 11,4%). With regard to SARS-CoV testing, material obtained from expectorate, nasopharyngeal suction, tracheal secretion, BAL or graft from pharynx was amplified using a RealStar® SARS-CoV-2 RT-PCR Kit RUO from Altona Diagnostics (Hamburg, Germany) adapted to a Roche flow system. The limit of detection was 50 copies of RNA. 24 of the patients were diagnosed before arrival to the hospital.

suPAR testing. suPAR was tested using the suPARnostic QT test (ViroGates, Denmark). The majority of tests took place at first day in hospital, but some were taken after 24 hours (0-24 hrs (355, 93,4%), 24-48 hrs (3, 0,8%), 2-4 days (5, 1,3%), 4+ days (17, 4,5%)).

### Results

The suPAR levels were as shown in Table 3 below.

TABLE 3

| Total | | | | | | | Precentile | | | | | | |
|---|---|---|---|---|---|---|---|---|---|---|---|---|---|
| Count | | | | | | | | | | 0.50 | | | |
| (N) | Missing | Min | Max | Mean | StDev | Sum | 0.50 | 0.10 | 0.25 | Median | 0.75 | 0.90 | 0.95 |
| 430 | 2 (0.5%) | 1.90 | 56.00 | 5.62 | 5.10 | 2.416,10 | 1.90 | 2.00 | 2.73 | 4.20 | 6.40 | 10.73 | 13.69 |

After 14 days of follow-up, we found the following: Still hospitalized (42, 10,1%), Discharged and alive (331, 79,8%), Discharged and died at home (9, 2,2%), Died at hospital (33, 8,0%), With regard to organ dysfunction, this was reported in 392 patients and the following was observed: No organ dysfunction (337, 86,0%), Kidney (14, 3,6%), Liver (9, 2,3%), Lungs (49, 12,5%), Heart (10, 2,6%). Comparing patients that did not develop organ failure with those that did revealed significantly higher baseline suPAR levels in those who developed organ failure (p<0.001).

We aimed to determine whether the suPAR level at admission was predictive of whether a patient would end up with intubation and mechanical ventilation (Respirator). At baseline, the doctors were asked to evaluate whether a patient was suited for intubation if needed during follow-up. Patients not suited were the ones thought to be too weak to survive intubation and mechanical ventilation, e.g. very old patients or patients with advanced cancer or chronic obstructive lung disease prior to SARS-CoV-2 infection. 32 of the non-suited patients received palliative care. 316 patients were deemed possible to have intensive care treatment and 76 were not. During follow-up, 26 patients of the suitable patients ended up requiring respirator.

suPAR at Baseline as Predictor of Need of Respirator in Suitable Patients During 14-Days of Follow-Up

A ROC curve of baseline suPAR against the outcome of ending in a respirator during follow-up is shown in ROC curve forming part of the Figures. The area under the curve was 0,895 (p<0.001).

The Youden index was of suPAR (optimal sensitivity and specificity was 4.75 ng/ml. This provided a very high negative predictive value of 0,995.

Table 4 showing sensitivity, specificity, positive predictive value (ppv) and negative predictive value (npv)

| threshold | specificity | sensitivity | ppv | npv |
|---|---|---|---|---|
| 4.75 | 0.703 | 0.961 | 0.225 | 0.995 |

Table 5 showing that 1/205 patients with suPAR below 4.75 ng/ml ended up in respirator and 25/111 with suPAR above ended up in respirator. The difference is highly significant (p<0.001).

| | <=4.75 | >4.75 |
|---|---|---|
| Not requiring respirator | 204 | 86 |
| Requiring respirator | 1 | 25 |

With a cut-off of 6 ng/ml, the following results are obtained:

TABLE 6

| threshold | specificity | sensitivity | ppv | npv |
|---|---|---|---|---|
| 6.0 | 0.814 | 0.7692 | 0.270 | 0.975 |

Thus, a patient with suPAR below 6 ng/ml at baseline has a 2,5% chance of ending up in a respirator, while a patient with suPAR above 6 ng/ml has a 27% chance of ending up in a respirator. The difference is highly significant (p<0.001)

TABLE 7

| | <=6 | >6 |
|---|---|---|
| Not requiring respirator | 240 | 50 |
| Requiring respirator | 6 | 20 |

In conclusion, we find that the baseline suPAR level is predictive of the risk of ending up in need of intubation and mechanical ventilation (respirator).

1. A method of assessing whether a subject who has COVID-19 symptoms and/or SARS-CoV-2 infection requires or is likely to require oxygen supplementation, particularly invasive ventilation (respirator), the method comprising determining whether the subject has a suPAR plasma level of over 4.75 ng/ml, preferably over 6 ng/ml.

2. A method according to claim 1 wherein the COVID-19 symptoms include one or more of a fever and a new continuous cough.

3. A method according to claim 1 or 2 wherein SARS-CoV-2 infection is diagnosed by a method comprising any of those disclosed in Table 1 above, preferably the RealStar® SARS-CoV-2 RT-PCR Kit RUO from Altona Diagnostics (Hamburg, Germany).

4. A method according to any of the preceding claims wherein the plasma level is determined directly by assaying a plasma sample or indirectly by assaying a serum or urine level.

5. A method according to any of the preceding claims wherein the suPAR level is determined by an immunodiagnostic method.

6. A method of determining the likelihood of death within a period of time of a subject who has COVID-19 symptoms and/or SARS-CoV-2 infection and is being treated with, or is being considered for treatment with, assisted respiration, the method comprising determining whether the subject has a suPAR plasma level of over 4.75 ng/ml, preferably over 6 ng/ml.

7. A method according to claim 6 wherein the assisted respiration is invasive ventilation.

8. A method of assessing whether a subject who has COVID-19 symptoms and/or SARS-CoV-2 infection may be discharged from hospital or not admitted to hospital, the method comprising determining the subject's suPAR level, such that
(i) a subject having a suPAR plasma level of below 4.75 ng/ml is discharged or is not admitted, and
(ii) a subject having a suPAR plasma level of 6 ng/ml or above is not discharged or is admitted.

9. A method according to claim 8 wherein a subject having a suPAR plasma level of 6 ng/ml or above is additionally considered to be likely to require oxygen supplementation, particularly invasive ventilation (respirator).
